(19) United States
(12) Patent Application Publication
(10) Pub. No.: US 2013/0158321 A1
(43) Pub. Date: Jun. 20, 2013

(54) PROCESS FOR THE PRODUCTION OF OCTENES BY DIMERIZING ETHYLENE TO FORM BUTENES AND DIMERIZING THE BUTENES TO FORM OCTENES

(71) Applicant: IFP Energies nouvelles, Rueil-Malmaison Cedex (FR)

(72) Inventors: Helene OLIVIER-BOURBIGOU, Saint Genis-Laval (FR); Francois HUGUES, Vernaison (FR); Lionel MAGNA, Lyon (FR); Pierre-Alain BREUIL, Lyon (FR)

(73) Assignee: IFP ENERGIES NOUVELLES, Rueil-Malmaison Cedex (FR)

(21) Appl. No.: 13/716,590

(22) Filed: Dec. 17, 2012

(30) Foreign Application Priority Data
Dec. 20, 2011 (FR) 1103996

(51) Int. Cl. C07C 2/24

(52) U.S. Cl. CPC C07C 2/24
USPC 585/329

(57) ABSTRACT

The present invention describes a process for the production of octenes from ethylene by dimerizing ethylene to butenes and by dimerizing butenes to octenes.

The present invention describes a process for the production of octenes from ethylene by dimerizing ethylene to butenes and by dimerizing butenes to octenes.

### PRIOR ART CONCERNING THE PRODUCTION OF OCTENES

Various sources for the production of octenes exist:

i) extraction of an olefinic C8 cut from a polygas type process (Cat Poly™ process) using catalytically cracked C4;

ii) dimerizing n-butenes contained in a raffinate II by means of a Dimersol™ or Octol™ type process;

iii) dimerizing isobutene contained in a raffinate I by means of a process of the Selectopol™ or Dimer8™ or NextOctane™ or In Alk™ type.

Pathways i) and iii) result in the production of highly branched C8 olefins. The octenes produced by pathway ii), the dimerization of n-butenes, are of particular interest as they are less branched.

Those octenes produced by the Dimersol™ process from Axens (Institut Français du Petrole, Review, Vol. 37, N^{o} 5, September-October 1982, p 639) or the Octol™ process from Hüls AG (Hydrocarbon Processing, February 1992, p 45-46), which are obtained at advantageous prices, may in particular be transformed in good yields by a hydroformylation reaction then by hydrogenation to isononanols. Those C9 alcohols are primarily used for the synthesis of phthalate type plasticizers for PVC. Those alcohols provide polymers with particular properties: very good compatibility with the polymer chain, low volatility, low temperature flexibility, high temperature stability, processability, etc.

In that pathway ii), the feeds used are C4 cuts. The C4 cuts are available in large quantities in refineries and petrochemicals sites. They are produced by conventional processes such as fluid catalytic cracking or steam cracking of naphtha. Those C4 cuts typically contain olefinic compounds such as n-butenes (1-butene and 2-butenes), isobutene and also paraffins (isobutane and n-butane), dienes (such as 1,3-butadiene) as well as traces of acetylenic impurities. Table 1 shows the compositions of C4 cut types obtained from fluid catalytic cracking and steam cracking.

When carried out on such olefinic C4 cuts, dimerization processes necessitate a) the elimination of butadiene by solvent extraction or selective hydrogenation to butenes, and b) the separation or transformation of isobutene.

TABLE 1
Example of C4 cut type composition

| | Typical | Composition |
|---|---|---|
| | composition | (wt %) |
| | (wt %) FCC | Steam cracking |
| | (after selective | (after selective |
| | hydrogenation) | hydrogenation) |
| n-butane | 11 | 5 |
| 1-butene | 13 | 28 |
| 2-butene | 26 | 20 |
| isobutene | 15-25 | 45-50 |
| isobutane | 35 | 2 |
| butadiene | 0.5 | 1 |

Industrially, the separation of butadiene is carried out, for example, by liquid-liquid extraction with a solvent (N-methyl-2-pyrrolidone, DMF, . . . ) and/or by selective hydrogenation in order to obtain a mixture of isobutenes, n-butenes and butanes. The isobutene is then separated from the other C4 compounds. The isobutene is separated by complicated routes, transforming them using processes such as etherification with methanol or selective oligomerization or polymerization, resulting respectively and most typically in MTBE (methyl-tert-butyl-ether), DIB (diisobutene) or PIB (polyisobutene). After elimination of the isobutene, only the n-butenes and butanes (raffinate II) are left. The publication Petroleum Technology Quarterly, autumn 1999, p 141 describes that concatenation of steps for the production of octenes from C4 cuts.

One supplemental possibility for transforming C4 olefins into octenes is the concatenation of Dimersol™-Difasol™ processes or the direct use of the two-phase Difasol™ process as described in the article Oil Gas Eur. Mag. 2005, 2, p 83.

However, it is known (IFP Review, volume 37, No 5, September-October 1982, p 639) that isobutene, a branched olefin, is capable of reacting cationically during the dimerization process, leading to the formation of heavy oligomers. Polyunsaturated hydrocarbons (dienes) are dimerization poisons. The nitrogen-containing, oxygen-containing or sulphur-containing polar compounds (which may derive from steps for extraction of butadiene by polar solvents or etherification steps to separate the isobutene) are also catalyst poisons, even though they are present in trace amounts.

As a consequence, using such C4 cuts necessitates purification steps. The traces of impurities cited above are generally never completely eliminated.

### SUMMARY OF THE INVENTION

The present invention describes a process for the production of octenes from ethylene, employing the following steps:

a) dimerizing ethylene to butenes in the presence of a catalyst;

b) dimerizing the butenes obtained in step a) to octenes in the presence of a catalyst.

### Advantage of the Invention

It has now been discovered that it may be advantageous to form butenes for dimerization by dimerizing ethylene.

One of the premier advantages of the process of the invention over processes using a fluid catalytic cracking C4 cut or steam naphtha cracking cut is that there is no need to carry out any purification. In particular, isobutene does not have to be eliminated, because the n-butenes obtained from dimerization do not contain any of it. In fact, the process of the present invention can be carried out without a purification step between the ethylene dimerization step and the butenes dimerization step.

In addition, the butenes obtained by the dimerization of ethylene are free of butane, which has the advantage of not diluting the olefinic reagent in the feed for the dimerization process.

Furthermore, the butenes formed by dimerization do not contain oxygen-containing impurities. Thus, they do not have to be purified. This absence is particularly advantageous when carrying out the Dimersol™ process, the Difasol™ process alone or in combination with the Dimersol™ process, or the Octol™ process.

Another advantage of the process of the invention is that ethylene is used (for example obtained from steam cracking of ethane), and so there are no restrictions due to the availability of a C4 cut (furthermore, another advantage is that it is possible to use a source of ethylene other than of fossil origin). In particular, the ethylene may be obtained from renewable sources, for example from the dehydration of ethanol, or from any other source. The ethylene is thus obtained from a fossil source and/or from a renewable source.

### DESCRIPTION OF THE PROCESS

The first step in the process of the invention comprises dimerizing ethylene to butenes in the presence of a catalyst.

### a/ Dimerization of Ethylene to Butenes

Ethylene can be dimerized to butenes using any catalytic process which is known in the art, preferably including those which lead to a high selectivity for dimers (1-butene or 2-butene).

In a first variation, ethylene dimerization is carried out in the presence of a titanium-based catalyst. Such catalysts have been described in patent FR 2 552 079 (U.S. Pat. No. 4,615,998). A homogeneous catalytic system is used which can selectively produce 1-butene from ethylene under mild conditions. The catalytic system is composed of an alkyl titanate, an ether type organic additive and an aluminium compound with formula AlR₃ or AlR₂H, in which each of the residues R is a hydrocarbyl radical, preferably alkyl containing 2 to 6 carbon atoms, for example triethylaluminium, tripropylaluminium, triisobutylaluminium, the hydride of diisobutylaluminium or trihexylaluminium. The alkyl titanates used have general formula Ti(OR′)₄ in which R′ is a linear or branched alkyl radical containing 2 to 8 carbon atoms, for example tetraethyl titanate, tetraisopropyl titanate, tetra-n-butyl titanate or tetra-2-ethylhexyl titanate. The organic ether type additive and the alkyl titanate are used in a molar ratio of 1:0.5 to 10:1. The dimerization reaction is carried out at a temperature in the range 20° C. to 70° C. The pressure is preferably 0.5 to 8 MPa.

The Alphabutol™ process (described in the review Hydrocarbon Processing 1984, pp 118-120) is based on this technology and results in the industrial production of 1-butene using a simple layout.

In a second variation, ethylene is dimerized in the presence of a catalytic system composed of a nickel compound and an aluminium compound. Such catalytic systems are described in patent U.S. Pat. No. 3,485,881. That process consists of dimerizing mono-olefins (including ethylene) by bringing them into contact with a catalytic system formed by a mixture of a first compound selected from the group comprising the following formulae:

(R₃P)₂NiY₂, (R₃PO)₂NiX₂, (R₃AsO)₂NiX₂, (pyridine)₂NiX₂, (bipyridine)NiX₂, (phenanthroline)NiX₂ and a complex formed by a bicyclic nitrogen-containing compound with NiX₂, where R is a hydrocarbon group containing up to 20 carbon atoms, X is a halogen, Y is selected from halogens or hydrocarbon groups as defined above and pyridine, bipyridine and phenanthroline which may or may not be substituted with one or more hydrocarbon groups, and a second compound represented by the formula R′_{z}AlX_{y}, where x and y are whole numbers in the range 1 to 3, R′ is a hydrocarbon group containing up to 20 carbon atoms and X is a halogen. The ratio between the first compound (Ni) and the second compound (Al) is in the range 1:0.5 to 1:20. The dimerization reaction is carried out at a temperature in the range −80° C. to 100° C. The pressure may be up to 13.8 MPa (2000 psig). Butenes are obtained, with a selectivity of more than 81%.

The very high purity of the butenes obtained from the ethylene dimerization processes of the invention mean that a prior purification step between the ethylene dimerization step and the butenes dimerization step can be dispensed with.

In fact, the effluent from the ethylene dimerization step principally contains butenes, as well as small quantities of unreacted ethylene and oligomers (in particular C6 and C8). These effluents in particular are free of any compounds that might poison the catalyst and the subsequent dimerization of the butenes.

In a first variation, the effluent obtained after the dimerization of ethylene, containing butenes, and after having optionally been separated from the catalyst, is sent as is to the butenes dimerization step. The oligomers present in the butenes are inert to dimerization of the butenes.

In a second variation, the effluent obtained after ethylene dimerization, containing butenes, and after having optionally been separated from the catalyst, is sent to a separation section in order to separate the butenes from the unreacted ethylene on the one hand (which is recycled to the ethylene dimerization reactor) and on the other hand from the oligomers formed during ethylene dimerization (hexenes and octenes). This separation may be carried out by distillation or flash separation.

The second step of the process of the invention comprises dimerizing the butenes obtained in step a) to octenes in the presence of a catalyst.

b/ Dimerization of n-butenes to octenes

The dimerization of butenes to octenes may be carried out using any catalytic process that is known in the art; preferably, they are selected from those which result in a high selectivity for dimers (straight chain octenes or slightly branched octenes).

In a first variation, dimerization of the butenes is carried out in the presence of a liquid phase catalytic system comprising a nickel compound and an aluminium compound. Such catalytic systems have been described for the Dimersol™ process sold by Axens. The various catalytic compositions described are used in the process of the invention.

As an example, patent FR 2 443 877 describes a composition comprising: at least one divalent nickel compound, at least one hydrocarbylaluminium dihalide with formula AlRₙX₃₋ₙ, in which R is a hydrocarbyl radical containing 1 to 12 carbon atoms, X is a chlorine or bromine atom, and n is a number in the range 0 to 1.

An improved catalytic composition is described in patent FR 2 794 038. It is obtained by bringing the following into contact in any order: at least one divalent nickel compound, with at least one hydrocarbylaluminium dihalide with formula AlRX₂ in which R is a hydrocarbyl radical containing 1 to 12 carbon atoms, X is a chlorine or bromine atom, and at least one organic Bronsted acid, preferably with a pKa of at most 3.

The divalent nickel compound used may be any compound which is soluble to more than 1 g per litre in a hydrocarbon medium, more particularly in the reagents and the reaction medium. Preferably, nickel carboxylates with general formula (R₁COO)₂Ni are used, where R₁ is a hydrocarbon residue, for example alkyl, cycloalkyl, alkenyl, aryl, aralkyl or alkaryl, containing up to 20 carbon atoms, preferably a hydrocarbon residue containing 5 to 20 carbon atoms. The radical R₁ may be substituted with one or more halogen atoms, hydroxyl groups, ketone, nitro, cyano or other groups which do not interfere with the reaction. The two radicals R₁ may also constitute an alkylene residue containing 6 to 18 carbon atoms. Non-limiting examples of nitrogen-containing compounds are the following divalent nickel salts: octoate, 2-ethyl hexanoate, decanoate, stearate, oleate, salicylate, and hydroxydecanoate. Preferably, nickel 2-ethyl hexanoate is used.

The Bronsted acid has formula HY, where Y is an organic anion, for example carboxylic, sulphonic or phenolic. Acids with a pKₐ at 20° C. which is a maximum of 3 are preferred, more particularly those which are also soluble in the nickel compound or in its solution in a hydrocarbon or other suitable solvent. A preferred class of acids comprises the group formed by halogenocarboxylic acids with formula R₂COOH in which R₂ is a halogenated alkyl radical, in particular those which comprise at least one halogen atom alpha to the —COOH group with a total of 2 to 10 carbon atoms. Preferably, a halogenoacetic acid with formula CXₚH₃₋ₚ—COOH is used in which X is fluorine, chlorine, bromine or iodine, where p is a whole number from 1 to 3. Examples which may be cited are trifluoroacetic, difluoroacetic, fluoroacetic, trichloroacetic, dichloroacetic and chloroacetic acids. These examples are not limiting; it is also possible to use arylsulphonic, or alkylsulphonic, fluoroalkylsulphonic acids, picric acid or nitroacetic acid. Preferably, trifluoroacetic acid is used.

Hydrocarbylaluminium dihalide compounds enriched in aluminium trihalide have general formula AlRₙX₃₋ₙ in which R is a hydrocarbyl radical containing 1 to 12 carbon atoms such as alkyl, aryl, aralkyl, alkaryl or cycloalkyl, X is a chlorine or bromine atom, and n is a number in the range 0 to 1. They are obtained by mixing a hydrocarbylaluminium dihalide compound with formula AlRX₂ in which R is a hydrocarbyl radical containing 1 to 12 carbon atoms such as alkyl, aryl, aralkyl, alkaryl or cycloalkyl and X is a chlorine or bromine atom, with an aluminium trihalide, AlX₃. The compounds in the following non-limiting list may be cited by way of example: dichloroethylaluminium enriched with aluminium chloride, the mixture having formula AlEt_{0.9}Cl_{2.1}, dichloroisobutylaluminium enriched with aluminium chloride, the mixture having formula AliBu_{0.9}Cl_{2.1}, dibromoethylaluminium enriched with aluminium bromide, the mixture having formula AliBu_{0.9}Cl_{2.1}.

Optionally, the components described above are mixed, in a solvent, at a controlled temperature and for a predetermined time; this constitutes a step termed pre-conditioning before using them in the reaction as described in patent U.S. Pat. No. 7,235,703. More precisely, this pre-conditioning of the catalytic composition consists of mixing the three components in a hydrocarbon solvent, for example an alkane or an aromatic hydrocarbon, or a halogenated hydrocarbon or, as is preferred, the olefins produced in the oligomerization reaction, with agitation and under an inert atmosphere, for example under nitrogen or under argon, at a controlled temperature in the range 0° C. to 80° C., preferably in the range 10° C. to 60° C., for a period of 1 minute to 5 hours, preferably 5 minutes to 1 hour. The solution obtained thereby is then transferred to the dimerization reactor under an inert atmosphere.

Another improved composition is described in patent U.S. Pat. No. 4,283,586. It comprises the nickel and aluminium compounds described above, along with an organic polyol such as ethylene glycol.

The Dimersol™ process is generally carried out at a temperature of −20° C. to 80° C., under pressure conditions such that the reagents are maintained at least mainly in the liquid phase or in a condensed phase.

The dimerization step may be carried out in a reactor with one or more reaction stages in series, the olefinic feed and/or the catalytic composition which has been pre-conditioned before being introduced continuously, either into the first stage or into the first and any other of the stages. At the outlet from the reactor, the catalyst may be deactivated, for example by injecting ammonia and/or an aqueous solution of sodium hydroxide and/or an aqueous solution of sulphuric acid. The unconverted olefins and any alkanes present in the feed are then separated from the oligomers by distillation.

In a second variation, the butenes dimerization is carried out in the presence of a catalytic system comprising a nickel complex or a mixture of nickel complexes in a two-phase liquid-liquid medium containing a medium with an ionic nature which is not or is only slightly miscible with the organic phase containing the octenes. Such a catalytic system is described in patent U.S. Pat. No. 5,104,840 (Difasol™ process).

The medium with an ionic nature comprises at least one salt with formula Q⁺A⁻, in which Q⁺ is a quaternary ammonium or phosphonium cation or a mixture of the two or a lithium cation, and A⁻ is a coordinating or non-coordinating anion selected from the group formed by halogenoaluminates, organohalogenoaluminates, organogallates, organohalogenogallates or a mixture of at least two of these compounds.

Thus, the catalysts that may be used are any complexes of nickel, apart from nickel complexes solvated by compounds containing active protons such as water, alcohols, organic acids or ammonia. Examples of such complexes which may be cited are nickel acetylacetonate, complexes of nickel halides or sulphates such as (NiCl₂, 2 pyridine), (NiCl₂, 2PPr₃), (NiBr₂, 2PBu₃), (NiSO₄, 2PBu₃), nickel octoate or nickel alcoholates. It is also possible to use nickel compounds described in French patent FR 2 611 700, which comprise at least one nickel-carbon or nickel-hydrogen bond, or which have a valency of less than 2; examples of such compounds which may be cited are nickel biscyclooctadiene, methallylnickel chloride, bisallylnickel, allylnickel chloride and allylnickel bromide.

The catalytic reaction in a two-phase medium is carried out at a temperature below +100° C., for example in the range −50° C. to +100° C., the pressure being kept in the range 0.01 to 20 MPa.

Compared with an entirely homogeneous liquid system such as that described in the Dimersol™ process, using a two-phase system means that the reaction volume as well as the consumption of catalyst can be reduced and the selectivity for dimers can be increased.

In a third variation, the two-phase Difasol™ system may also be employed after a first homogeneous Dimersol™ step, as described in patents U.S. Pat. No. 6,444,866 and U.S. Pat. No. 6,203,712. In this case, dimerization of the butenes comprises at least two successive steps, the first being carried out in the presence of a liquid phase catalytic system comprising a nickel compound and an aluminium compound, the second being carried out in the presence of a catalytic system comprising a nickel complex or a mixture of nickel complexes in a liquid-liquid two-phase medium containing a medium with an ionic nature which is not or is only slightly miscible with the organic phase containing the reaction products (octenes).

The catalytic composition of the first step, in homogeneous liquid phase, is preferably as follows: the catalyst is a compound of nickel or a mixture of nickel compounds, the co-catalyst is an alkylaluminium or a mixture of alkylaluminium compounds or a halogenoalkylaluminium or a mixture of halogenoalkylaluminium compounds, the Bronsted acid is a halogenoacetic acid or a mixture of halogenoacetic acids and the optional catalyst additive may be a compound with an acid nature, the anion corresponding to that acid, a carboxylic acid ester, an epoxy compound or a phosphine.

The catalytic composition of the second step is identical to that described above for the Difasol™ process. The polar phase of the second step may also be an ionic medium which is not miscible with the organic phase containing no catalyst; the catalyst of the second step in a liquid-liquid two-phase medium is then the catalyst used in the first step. The catalyst is then introduced into the reactor with the effluent leaving the reactor for the first step.

The operating conditions for the two steps are identical to those indicated above for the Dimersol™ and Difasol™ process respectively.

In a fourth variation, the butenes dimerization is carried out in the presence of a heterogeneous catalyst containing oxides of nickel, silicon and aluminium. Such a catalyst is described in patents U.S. Pat. No. 5,177,282 and DD 160037 (the Octol™ process).

This variation consists of dimerizing butenes with a heterogeneous catalyst based on nickel at temperatures in the range 0° C. to 200° C. and pressures in the range 0.1 to 7 MPa.

Preferably, the butenes are dimerized in a fixed bed reactor over a heterogeneous catalyst containing oxides of nickel, silicon and aluminium. The butenes are dimerized in the liquid phase.

The catalyst preferably contains oxides of nickel, silicon and aluminium. Highly preferably, the catalyst contains in the range 0.1% to 10% by weight of alumina and in the range 0.1% to 10% by weight of nickel oxide, and silica.

In a fifth variation, the butenes dimerization is carried out in the presence of a heterogeneous catalyst containing nickel, as well as chlorine or sulphur, deposited on alumina.

Such a process is described in patents EP346187, EP272970 and FR2641477. It leads to the selective formation of dimers from light olefins.

In this variation, the butenes dimerization may be carried out in the liquid phase in the presence of at least one olefin dimerization catalyst comprising chlorine and nickel (with proportions which are preferably non-stoichiometric) deposited on alumina, the quantity of nickel in said catalyst generally being in the range 0.5% to 15% by weight and the quantity of chlorine in said catalyst normally being in the range 0.4% to 15% by weight. The process is carried out at a temperature in the range 10° C. to 150° C.; the pressure is maintained such that the reaction mixture is in the liquid state.

In the case of a heterogeneous catalyst containing nickel and sulphur deposited on alumina, the catalytic composition is obtained by operating as follows:

a) bringing the alumina into contact with a source of sulphate ions and a source of nickel ions in aqueous or organic solution, the proportions of sulphate ions and of nickel ions being selected such that said ions are introduced into the alumina in proportions corresponding to a S:Ni atomic ratio of less than 1:1, preferably in the range 0.1:1 to 0.95:1;

b) eliminating the solvent from the alumina which has absorbed said ions in the above proportions; and

c) heating to between 300° C. and 800° C. in a non-reducing atmosphere.

The very high purity of the butenes obtained from the ethylene dimerization processes of the invention means that a purification step between the ethylene dimerization step and the butenes dimerization step can be dispensed with.

In fact, purification steps are recommended when the C4 olefins originate from refining. As an example, in the case of heterogeneous dimerization processes (in the nickel case), the olefins of the feed are passed over a molecular sieve with a pore diameter in the range 4 to 15 angstroms to capture catalyst poisons: water, alcohols, nitrogen-containing compounds, sulphur-containing compounds or halogenated compounds.

The products of the present process (octenes) may have an application, for example, as a component of automobile fuels and/or as a feed for a hydroformylation process for the synthesis of aldehydes and alcohols.

### EXAMPLE

The following example illustrates the invention without in any way limiting its scope.

### Step a). Production of 1-Butene by Dimerizing Ethylene

A 3.2 t/h stream of pure ethylene obtained from an ethane steam cracking unit was sent to a reactor operating essentially in the liquid phase into which 10.8 kg/h of a 6% by weight solution of triethylaluminium in hexane was sent, along with 1.3 kg/h of a liquid titanium-based catalyst. The reactor was held at a temperature of 50° C. to 55° C. by continuously removing the heat of reaction by means of a recirculation loop provided with a cold water exchanger and at a pressure of 2.4 MPa. Under these conditions, the ethylene per pass conversion was 85%. The effluent leaving the reactor was neutralized with a base and the catalyst was separated from the reaction products. The products were then separated, by two distillation columns, into:
unreacted ethylene, which was recycled to the reactor inlet;
a C4 cut, essentially constituted by pure 1-butene with a purity of more than 99.5%, at a flow rate of 2.9 t/h;
a C6 cut constituted by a mixture of isomers primarily constituted by 2-ethyl-1-butene and 3-methyl-1-pentene at a flow rate of 0.22 t/h.

### Step b). Production of Octenes by Dimerizing 1-Butene

The C4 cut obtained in step a) was sent to a unit constituted by three reactors disposed in series, operating in the liquid phase, all equipped with a recirculation loop provided with a cold water exchanger in order to continuously evacuate the heat of reaction and maintain the temperature between 45° C. and 50° C.

The C4 cut (2.9 t/h) was injected into the first reactor, as well as 11.6 kg/h of a 50% by weight solution of dichloroalkylaluminium in a paraffinic C6-C8 gasoline cut and 1.9 kg/h of liquid nickel-based catalyst.

The effluent from the first reactor was sent as a whole to the second; the effluent from the second reactor was sent to the third reactor.

Under these conditions, the butene conversion was 81%.

The effluent leaving the third reactor was neutralized with a base and the catalyst was separated into the reaction products; the products were separated, by two distillation columns, into:
a C4 cut at a flow rate of 0.56 t/h, containing unreacted butene—which was sent to the LPG (liquefied petroleum gas) pool;
a C8 cut at a flow rate of 1.95 t/h, constituted by a mixture of isomers (n-octenes: 5%; methylheptenes: 65%, dimethylhexenes: 30%);
a C12+ cut at a flow rate of 0.39 t/h, mainly constituted by dodecenes.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding French application No. 11/03.996, filed Dec. 20, 2011, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A process for the production of octenes starting from ethylene, employing the following steps:
a) dimerizing ethylene to butenes in the presence of a catalyst;
b) dimerizing the butenes obtained in step a) to octenes in the presence of a catalyst.

2. A process according to the preceding claim 1, in which the effluent obtained after ethylene dimerization, containing butenes, is sent as is to the butenes dimerization step.

3. A process according to claim 1, in which the effluent obtained after ethylene dimerization, containing butenes, is sent to a separation section in order to separate the butenes from unreacted ethylene on the one hand, and on the other hand from the oligomers formed during ethylene dimerization.

4. A process according to the claim 1, in which the ethylene dimerization is carried out in the presence of a titanium-based catalyst.

5. A process according to claim 4, in which the titanium-based catalyst is composed of an alkyl titanate, an organic ether type additive and an aluminium compound with formula AlR₃ or AlR₂H in which each of the residues R is a hydrocarbyl radical, and the ether type additive and the alkyl titanate are employed in a molar ratio of 1:0.5 to 10:1.

6. A process according to claim 1, in which the ethylene dimerization is carried out in the presence of a catalytic system composed of a nickel compound and an aluminium compound.

7. A process according to claim 6, in which the catalytic system is a mixture of a first compound selected from the group comprising the following formulae: (R₃P)₂NiY₂, (R₃PO)₂NiX₂, (R₃AsO)₂NiX₂, (pyridine)₂NiX₂, (bipyridine)NiX₂, (phenanthroline)NiX₂ and a complex formed by a bicyclic nitrogen-containing compound with NiX₂, where R is a hydrocarbon group containing up to 20 carbon atoms, X is a halogen, Y is selected from halogens or hydrocarbon groups as defined hereinabove and pyridine, bipyridine and phenanthroline which may or may not be substituted with one or more hydrocarbon groups, and a second compound represented by the formula R′_{z}AlX_{y}, where x and y are whole numbers in the range 1 to 3, R′ is a hydrocarbon group containing up to 20 carbon atoms and X is a halogen.

8. A process according to claim 1, in which the butenes dimerization is carried out in the presence of a catalytic system in the liquid phase comprising a nickel compound and an aluminium compound.

9. A process according to claim 8, in which the catalytic system is composed of at least one divalent nickel compound with at least one hydrocarbylaluminium dihalide with formula AlRX₂ in which R is a hydrocarbyl radical containing 1 to 12 carbon atoms, X is a chlorine or bromine atom, and at least one organic Bronsted acid, preferably with a pKₐ of at most 3.

10. A process according to claim 1, in which the butenes dimerization is carried out in the presence of a catalytic system composed of a nickel complex or a mixture of nickel complexes in a two-phase liquid-liquid medium containing a medium with an ionic nature which is not or is only slightly miscible with the organic phase containing the octenes.

11. A process according to claim 1, in which the butenes dimerization comprises at least two successive steps, the first being carried out in the presence of a catalytic system in the liquid phase comprising a nickel compound and an aluminium compound, the second being carried out in the presence of a catalytic system comprising a nickel complex or a mixture of nickel complexes in a two-phase liquid-liquid medium containing a medium with an ionic nature which is not or is only slightly miscible with the organic phase containing the octenes.

12. A process according to claim 1, in which the butenes dimerization is carried out in the presence of a heterogeneous catalyst containing oxides of nickel, silicon and aluminium.

13. A process according to claim 1, in which the butenes dimerization is carried out in the presence of a heterogeneous catalyst containing nickel, as well as chlorine or sulphur, deposited on alumina.

14. A process according to claim 1, in which the ethylene is obtained from a fossil source and/or from a renewable source.

15. A process according to claim 1, in which the ethylene is obtained from ethanol dehydration.
